(19) United States
(12) Patent Application Publication
(10) Pub. No.: US 2026/0325714 A1
(43) Pub. Date: Oct. 22, 2026

(54) Compositions and Methods for Improving Cartilage Formation

(71) Applicants: Emory University, Atlanta, GA (US); The United States Government as represented by the Department of Veterans Affairs, Washington, DC (US)

(72) Inventors: Jay M. Patel, Atlanta, GA (US); Sameh A. Labib, Atlanta, GA (US); David R. Myers, Atlanta, GA (US); Maddie Hasson, Atlanta, GA (US); Lorenzo M. Fernandes, Atlanta, GA (US)

(21) Appl. No.: 19/101,448

(22) PCT Filed: Aug. 4, 2023
(86) PCT No.: PCT/US2023/029495

§ 371 Date: Feb. 5, 2025

Related U.S. Application Data
(60) Provisional application No. 63/395,416, filed on Aug. 5, 2022.

(51) Int. Cl. A61L 27/24; A61L 27/36; A61L 27/44; A61L 27/54

(52) U.S. Cl. CPC A61L 27/24; A61L 27/365; A61L 27/44; A61L 27/54; A61L 2300/216; A61L 2300/254; A61L 2300/414

(57) ABSTRACT

This disclosure contemplates methods of improving the growth of damaged cartilage. In certain embodiment, this disclosure relates to methods performing microfracture and applying components reported herein to provide marrow stimulation with improved cartilage regeneration. In certain embodiments, compositions and materials comprise fasudil optionally in combination with thrombin, aprotinin, and transforming growth factor-beta.

### CROSS-REFERENCE TO RELATED APPLICATIONS

This application claims the benefit of U.S. Provisional Application No. 63/395,416 filed Aug. 5, 2022. The entirety of this application is hereby incorporated by reference for all purposes.

### BACKGROUND

Microfracture is a commonly practiced procedure for cartilage repair that induces the migration of bone marrow mesenchymal stem cells (MSCs) to a site of damaged cartilage. The procedure typically involves drilling small holes into the subchondral bone marrow space which underlies regions of damaged cartilage. Bleeding at the defect site results in the formation of a blood clot. The clot typically contains multipotent MSCs from the bone marrow, which have the potential to differentiate into chondroblasts and chondrocytes. However, the cartilage formed is often unhealthy and fibrotic. Fibrotic cartilage is non-durable and functionally problematic in the long-term. Hyaline cartilage is durable and provides shock absorption and lubrication in diarthrodial joints (articular cartilage). Inducing the formation of hyaline like cartilage rather than fibrotic cartilage remains a challenging clinical problem. Therefore, there remains a need to identify improved methods for repairing cartilage damage.
Fan et al. report a TGF-β3 immobilized PLGA-gelatin/chondroitin sulfate/hyaluronic acid hybrid scaffold for cartilage regeneration. J Biomed Mater Res, 2010, 95A: 982-992.
Hoffman et al. report articular cartilage repair using marrow stimulation augmented with a viable chondral allograft. Case Rep Orthop, 2015, 617365.
Chu et al. report methods for repairing cartilage damage using SOX9. US Pub. App. No. 2017/0197011.
Martin et al. report emerging therapies for cartilage regeneration. NPJ Regenerative Medicine, 2019, 4:1.
Patel et al. report bioactive factors for cartilage repair and regeneration. Acta Biomater, 2019, 93222-238.
Martin et al. report nanofibrous hyaluronic acid scaffolds delivering TGF-β3 and SDF-1α for articular cartilage repair. Acta Biomater, 2021, 126:170-182.

References cited herein are not an admission of prior art.

### SUMMARY

This disclosure contemplates methods of improving the growth of damaged cartilage. In certain embodiment, this disclosure relates to methods performing microfracture and applying components reported herein to provide marrow stimulation with improved cartilage regeneration.

In certain embodiments, this disclosure contemplates methods and composition for use in the maintenance of cartilage volume, maintenance of repair volume, limiting contraction, inhibiting fibrosis, and priming of eventual cartilage regeneration. In certain embodiments, this disclosure contemplates supplementation of a bone marrow stem cell environment with structural components, remodeling components, cell contractility components, and optionally TGF-β or other chondrogenic stimuli which can be delivered in a liquid solution and/or within a scaffold (collagen, fibrin, chondral allograft).

In certain embodiments, this disclosure relates to methods of repairing damaged cartilage comprising administering to a subject in need thereof an effective amount of a composition having a structural component such as fibrinogen, thrombin, genipin, calcium salts, or combinations thereof, a remodeling component such as aprotinin, tranexamic acid, aminocaproic acid, factor XIII, or combinations thereof, and a cell contractility component such as fasudil, cytochalasin D, netarsudil, ripasudil, 4-(1-aminoethyl)-N-(pyridin-4-yl)cyclohexane-1-carboxamide (Y27632), or combinations thereof, at the area of desired cartilage growth to generate the formation, growth, repair of cartilage, or other tissue replacement.

In certain embodiments, this disclosure relates to methods of repairing or regenerating cartilage, minimizing the formation of fibrous tissue after microfracture, stimulating the formation of hyaline like cartilage, stimulating marrow, or preventing microfracture clot contraction after microfracture comprising puncturing a bone surface on an area of desired cartilage growth or repair at a sufficient depth to expose bone marrow containing mesenchymal stromal cells which migrate into the bone surface or cartilage defect, and implanting or injecting a composition reported herein at the area of desired cartilage growth in an amount effective to generate the formation, growth, or repair of cartilage or other tissue.

In certain embodiments, this disclosure relates to methods of repairing damaged cartilage comprising puncturing a bone surface on an area of desired cartilage growth at a sufficient depth to expose bone marrow containing mesenchymal stromal cells which migrate into the bone surface or cartilage defect and administering to a subject in need thereof an effective amount of fasudil at the area of desired cartilage or other tissue growth.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting a cell contractility inhibitor such as fasudil or other composition as reported herein at the area of desired cartilage growth in an amount effective to generate the formation, growth, or repair of cartilage or other tissue replacement.

In certain embodiments, this disclosure relates to methods of repairing damaged cartilage comprising administering to a subject in need thereof an amount effective of a composition disclosed herein, such as a composition comprising fasudil, at the area of desired cartilage growth. In certain embodiments, a fasudil is administered in combination with thrombin and/or aprotinin. In certain embodiments, fasudil is administered in combination with transforming growth factor-beta (TGF-β, TGF-β1, TGF-β3, or combinations thereof).

In certain embodiments, administering is injecting a liquid solution or implanting a matrix comprising fasudil or other components disclosed herein and transforming growth factor-beta. In certain embodiments, the liquid solution further comprises thrombin, aprotinin, factor XIII, factor XIIIα, fibrinogen, fibronectin, plasminogen, stromal cell-derived factor 1 alpha (SDF-1α), or combinations thereof. In certain embodiments, the matrix is a collagen matrix or a fibrin matrix. In certain embodiments, the matrix further comprises thrombin, aprotinin, factor XIII, factor XIIIα, fibrinogen, fibronectin, plasminogen, stromal cell-derived factor 1 alpha (SDF-1α), or combinations thereof.

In certain embodiments, this disclosure relates to methods of minimizing the formation of fibrous tissue after microfracture comprising puncturing a bone surface on an area of desired cartilage growth at a sufficient depth to expose bone marrow containing mesenchymal stromal cells which migrate into the bone surface or cartilage defect and administering an effective amount of fasudil, or other composition as reported herein at the area of desired cartilage growth. In certain embodiments, administering fasudil is in combination with thrombin and/or aprotinin. In certain embodiments, administering fasudil is in combination with transforming growth factor-beta.

In certain embodiments, administering is injecting a liquid solution or implanting a matrix. In certain embodiments, the liquid solution or matrix further comprises thrombin, aprotinin, transforming growth factor-beta, factor XIII, factor XIIIα, fibrinogen, fibronectin, plasminogen, stromal cell-derived factor 1 alpha (SDF-1α), a bone morphogenetic proteins (BMP, BMP-2, BMP-4, BMP-7), insulin-like growth factor 1 (IGF-1), basic fibroblast growth factor (bFGF), insulin, dexamethasone, kartogenin, a recombinant vector encoding SOX9, mesenchymal stromal cells (MSCs) comprising a recombinant vector encoding SOX9, or combinations thereof.

In certain embodiments, the matrix is a collagen matrix or a fibrin matrix. In certain embodiments, the matrix further comprises thrombin, aprotinin, transforming growth factor-beta, factor XIII, factor XIIIα, fibrinogen, fibronectin, plasminogen, stromal cell-derived factor 1 alpha (SDF-1a), Bone morphogenetic proteins (BMPs, BMP-2, BMP-4, BMP-7), insulin-like growth factor 1 (IGF-1), and basic fibroblast growth factor (bFGF), insulin, dexamethasone, kartogenin, recombinant vector encoding SOX9, MCSs comprising a recombinant vector encoding SOX9, or combinations thereof.

In certain embodiments, this disclosure relates to pharmaceutical compositions comprising of cartilage growth compositions disclosed herein and a pharmaceutically acceptable excipient. In certain embodiments, this disclosure relates to pharmaceutical compositions comprising fasudil and transforming growth factor-beta. In certain embodiments, this disclosure relates to pharmaceutical compositions comprising of fasudil, thrombin and/or aprotinin. In certain embodiments, this disclosure relates to pharmaceutical compositions comprising of fasudil and transforming growth factor-beta, thrombin, and/or aprotinin.

In certain embodiments, the pharmaceutical composition is in the form of an injectable solution. In certain embodiments, the pharmaceutical composition is in the form of a gel or polymer matrix. In certain embodiments, the polymer matrix is a sponge, hydrogel, collagen matrix or a fibrin matrix. In certain embodiments, the matrix is a biodegradable matrix. In certain embodiments, the matrix comprises poly(lactic-co-glycolic acid) (PLGA), gelatin, chondroitin sulfate, hyaluronic acid, or combinations thereof.

### BRIEF DESCRIPTION OF THE SEVERAL VIEWS OF THE DRAWINGS

FIG. 1 illustrates multi-scale microfracture (MFx). A surgical awl is used to puncture the subchondral bone recruiting marrow to clot within the defect. Marrow cells within the fibrin mesh can densify the matrix, experience cytoskeletal changes, and deposit new matrix, all of which are interrelated and may contribute to fibrosis.

FIG. 2 illustrates that using chondrogenic therapies alone result in “improved fibrocartilage”. However, it is ideal to include drivers to improve cartilage regeneration and limit fibrosis.

FIG. 3A shows MFx clot contraction with example histology images from a minipig model. MFx indicates varying degrees of clot contraction.

FIG. 3B shows data on the ICRS Score (tissue quality) versus defect fill (%), indicating a correlation between these variables.

FIG. 3C shows data indicating in vitro clot contraction is dependent upon thrombin concentration (mechanical control).

FIG. 3D shows a scan of a developing clot using bovine marrow cells indicating thrombin has a structural influence in contraction.

FIG. 4A shows data from clot contraction experiments using simulated clots treated with fasudil (FA) a Rho-ROCK inhibitor and an activator lysophosphatidic acid (LPA).

FIG. 4B shows data over the specified number of days in culture.

FIG. 4C shows data on collagen expression using fasudil in combination with TGF-β.

FIG. 5 shows data using aprotinin and TGF-β.

### DETAILED DESCRIPTION

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

An “embodiment” refers to an example of this disclosure, and the claims are not necessarily limited to such examples. The terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 5% or 10%. In all cases, where the term “about” has been used to describe a value, it should be appreciated that this disclosure also supports the exact value.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, have the meaning ascribed to them in U.S. Patent law in that when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

As used herein, the term “subject” refers to any animal, preferably a human patient, livestock, or domestic pet. In certain embodiments, the subject is a human subject of any age or a patient 2, 12, 16, or 20 years old or older. In certain embodiments, the subject is a human subject 55 or 65 years old or older. In certain embodiments, the subject is a human subject greater than 55, 60, 65, or 70 years of age.

“Fasudil” refers to the compound 5-((1,4-diazepan-1-yl)sulfonyl)isoquinoline or salts thereof. Fasudil HCl is reported to be a cyclic nucleotide-dependent protein kinase inhibitor and Rho-associated kinase inhibitor.

“Fibrinogen” refers to a combination of proteins that includes fibrinogen alpha, fibrinogen beta, fibrinogen gamma. Human reference sequences have the following UniProt accession numbers: P02671, P02675, and P02679. Together these proteins naturally polymerize to form an insoluble fibrin matrix. Fibrinogen functions in hemostasis as a component of blood clots. It is reported that the gamma-chain carries the main binding site for the platelet receptor. Fibrinogen from human plasma can be purchased commercially as a soluble dimer with a molecular weight of about 340 kDa.

“Thrombin,” also known as “prothrombin” or “activated factor IIα” refers to a procoagulant protein that cleaves and converts fibrinogen to fibrin which functions in blood homeostasis, inflammation, and wound healing. A human reference protein sequence has the following UniProt accession number P00734. Human forms of recombinant thrombin are commercially available with a molecular weight of about 37 kDa.

“Aprotinin” also known as “bovine basic pancreatic trypsin inhibitor (BPTI),” “Kunitz inhibitor” and “trypsin-kallikrein inhibitor” refers to an inhibitor of multiple serine proteases such as trypsin, plasmin, kallikrein, and activated factor XII. A reference protein sequence has the following UniProt accession number P00974. Recombinant forms are commercially available with a molecular weight of about 10 kDa.

“Transforming growth factor-beta” refers to a family of cytokines found in many human tissues which regulates proliferation, differentiation, adhesion, migration in many cell types. Three isoforms of TGF-βhave been identified: TGF-β1, TGF-β2 and TGF-β3. Transforming growth factor-β1 (TGF-β1) is found in mammalian platelets and many other tissues, typically present at higher level than the others. The TGF-β1 active form is composed of a TGF-β1 homodimer. A latent form is composed of a TGF-β1 homodimer, a LAP homodimer, and a latent TGF-β1 binding protein. The active form of recombinant human TGF-1 is commercially available as a disulfide linked homodimer with a molecular weight of about 25 kDa, with a sequence associated with UniProt accession number P01137. TGF-β3 is commercially available as a disulfide linked homodimer with a molecular weight of about 25 kDa with a sequence associated with a UniProt accession number P10600.1.

“SOX9” refers to a Sox (Sry-type HMG box) family protein and has been identified as a “master regulator” of the chondrocyte phenotype. Effects of SOX9 on MSCs are reported to include stimulating proliferation and promoting differentiation into chondrocytes. The amino acid sequence of human SOX9 protein is provided in the National Center for Biotechnology Information (NCBI) database with GenBank No.: CAA86598.1.

The term “mesenchymal stromal cells” and “MSCs” and the like refer to natural or an isolated subpopulation of fibroblast or fibroblast-like nonhematopoietic cells with properties of plastic adherence and capable of in vitro differentiation into cells of mesodermal origin which may be derived from bone marrow, adipose tissue, umbilical cord (Wharton's jelly), umbilical cord perivascular cells, umbilical cord blood, amniotic fluid, placenta, skin, dental pulp, breast milk, and synovial membranes. MSCs have a clonogenic capacity and can differentiate into several cells of mesodermal origin, such as adipocytes, osteoblasts, chondrocytes, skeletal myocytes, or visceral stromal cells. The term, “mesenchymal stem cells” is intended to include the cultured (self-renewed) progeny of primary mesenchymal stromal cell populations.

Bone marrow derived mesenchymal stromal cells are typically expanded ex vivo from bone marrow aspirates to confluence. Certain mesenchymal stromal/stem cells share a similar set of core markers and properties. Certain mesenchymal stromal/stem cells may be defined as positive for CD105, CD73, and CD90 and negative or low for CD45, CD34, CD14, and have the ability to adhere to plastic. See Dominici et al. Minimal criteria for defining multipotent mesenchymal stromal cells. The International Society for Cellular Therapy position statement. Cytotherapy, 2006, 8(4): 315-7.

As used herein, the term “biodegradable” refers to a solid material that will be broken down by biological mechanisms such that metabolites will be formed and the molecular arrangement, e.g., a matrix, membrane, sponge, will not persist for over a long period of time, e.g., the molecular arrangement will be broken down by the body after a several weeks or months. In certain embodiments, it is contemplated that naturally occurring components in the material will integrate with the host tissues or fluids.

### Cartilage Repair Methods

Articular cartilage is often damaged by trauma or degenerative arthritis, and it has a limited capacity for regeneration. A common joint disorder is osteoarthritis which presents symptoms such as severe pain, swelling and clicking of joints. Treatment of osteoarthritis typically consists of palliative pain relief and physical therapy which do not change the disease course. Many progress to advanced stage and require joint replacement. A common cause of osteoarthritis is the progressive breakdown of articular cartilage, and ultimately leads to functional failure of synovial joints. In certain embodiments, contemplated herein are methods of repairing cartilage in a subject diagnosed with osteoarthritis by using a matrix or composition disclosed herein such as to stimulate progenitor cells or cartilage transplants or a tissue-engineered cartilage like tissue to differentiate into chondrocytes in situ. Also contemplated is the use of a matrix or composition disclosed herein in combination with autologous cartilage transplants containing chondrocytes.

In certain embodiments, this disclosure relates to in situ regeneration of cartilage to repair defective articular cartilage. In certain embodiments, this disclosure contemplates methods using microfracture to induce migration of bone marrow mesenchymal stem cells (MSCs) to a site of a cartilage defect for the purpose of promoting cartilage production. In certain embodiments, the methods comprise the steps of drilling small holes into the subchondral bone marrow space which have regions of damaged cartilage thereby inducing bleeding at the defect site and allowing for the formation of a blood clot. It is contemplated that clot contains multipotent MSCs from the bone marrow or from exogenous sources with the ability to differentiate into chondroblasts and chondrocytes. In certain embodiments, it is contemplated that methods and composition disclosed herein are able to reduce the production fibrotic cartilage.

In certain embodiment, it is contemplated that compositions and methods disclosed herein are able to emulate the formation of hyaline cartilage or hyaline like cartilage which has improved durability with an extracellular matrix produced by chondrocytes consisting of collagen fibrils composed substantially of types II, IX, and XI collagen molecules, proteoglycans, and other matrix proteins. In certain embodiment it is contemplated that compositions and methods disclosed herein are able to emulate the formation of hyaline like cartilage which has improved durability with an extracellular matrix produced by chondrocytes that are absent of or have low levels of type I collagen.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting a cell contractility inhibitor such as fasudil at the area of desired cartilage growth in an amount effective to generate the formation or repair of cartilage or tissue replacement.

In certain embodiments, microfracture involves puncturing the bone with an awl, subchondral drilling, nanofracture (subchondral needling), or combinations thereof.

In certain embodiments, implanting or injecting fasudil is in combination with thrombin and/or aprotinin.

In certain embodiments, fasudil, thrombin, and aprotinin are contained in a matrix wherein the matrix optionally comprises collagen or a fibrin matrix optionally comprising factor XIII, factor XIIIα, fibrinogen, fibronectin, plasminogen, thrombin, aprotinin, TGF-β, or combinations thereof.

In certain embodiments, the thrombin is in an amount or concentration of greater than 0.5, 2, or 3 IU/mL, or between about 5 to 30 IU/mL, 10 to 50 IU/mL, 10 to 200 IU/mL, 100 to 300 IU/mL, 300 to 1000 IU/mL or 300 to 4000 IU/mL or of between about 1000 to 4000 IU/mL or 2000 to 5000 IU/mL.

In certain embodiments, methods further comprise implanting or injecting factor XIII, factor XIIIα, fibrinogen, fibronectin, plasminogen, thrombin, aprotinin, TGF-β, or combinations thereof at the area of desired cartilage growth, repair, or tissue replacement.

In certain embodiments, thrombin, fasudil, and/or aprotinin are implanted or injected in the absence or presence of transforming growth factor beta, dexamethasone, ascorbic acid or salt thereof, BMP-7, BMP-2, IGF-1, kartogenin, or combinations thereof.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting a Rho-kinase inhibitor or cell contractility inhibitor at the area of desired cartilage growth in an amount effective to generate the formation or repair of cartilage or tissue replacement.

In certain embodiments, implanting or injecting Rho-kinase inhibitor is in combination with thrombin and a fibrinolysis inhibitor. In certain embodiments, the Rho-kinase inhibitor is fasudil, ripasudil, netarsudil, 4-(1-aminoethyl)-N-(pyridin-4-yl)cyclohexane-1-carboxamide (Y27632), or salts thereof.

In certain embodiments, the fibrinolysis inhibitor is aprotinin, epsilon-aminocaproic acid, 4-aminomethylbenzoic acid, tranexamic acid, or salt thereof, or combinations thereof.

In certain embodiments, a Rho-kinase inhibitor, thrombin, and fibrinolysis inhibitor are contained in a matrix wherein the matrix optionally comprises collagen or a fibrin matrix optionally comprising factor XIII, factor XIIIα, fibrinogen, fibronectin, plasminogen, thrombin, aprotinin, TGF-β, or combinations thereof.

In certain embodiments, methods further comprise implanting or injecting factor XIII, factor XIIIα, fibrinogen, fibronectin, plasminogen, thrombin, aprotinin, TGF-β, or combinations thereof at the area of desired cartilage growth, repair, or tissue replacement.

In certain embodiments, thrombin, Rho-kinase inhibitor, and fibrinolysis inhibitor are implanted in the absence or presence of a chondrogenic agent at the area of desired cartilage growth, repair, or tissue replacement. In certain embodiments, the chondrogenic agent is transforming growth factor beta, dexamethasone, ascorbic acid or salt thereof BMP-7, BMP-2, IGF-1, kartogenin, or combinations thereof.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting fasudil or other Rho-kinase inhibitor and a chondrogenic agent(s) and optionally exogenous MSCs at the area of desired cartilage growth in an amount effective to generate the formation or repair of cartilage or tissue replacement. In certain embodiments, the chondrogenic agent is transforming growth factor beta, dexamethasone, ascorbic acid or salt thereof, BMP-7, BMP-2, IGF-1, kartogenin, or combinations thereof. In certain embodiments, implanting or injecting fasudil or Rho-kinase inhibitor and a chondrogenic agent is in combination with thrombin and aprotinin. In certain embodiments, fasudil or Rho-kinase inhibitor and chondrogenic agent are contained in a matrix, e.g., wherein the matrix optionally comprises collagen or a fibrin matrix optionally comprising factor XIII, factor XIIIα, fibrinogen, fibronectin, plasminogen, thrombin, aprotinin, TGF-β, or combinations thereof.

In certain embodiments, methods further comprise implanting or injecting factor XIII, factor XIIIα, fibrinogen, fibronectin, plasminogen, thrombin, aprotinin, TGF-β, or combinations thereof at the area of desired cartilage growth, repair, or tissue replacement.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting a Rho-kinase inhibitor and a chondrogenic agent at the area of desired cartilage growth in an amount effective to generate the formation or repair of cartilage or tissue replacement. In certain embodiments, the Rho-kinase inhibitor is fasudil, ripasudil, netarsudil, 4-(1-aminoethyl)-N-(pyridin-4-yl)cyclohexane-1-carboxamide (Y27632), or salt thereof.

In certain embodiments, the chondrogenic agent is transforming growth factor beta, dexamethasone, ascorbic acid or salt thereof BMP-7, BMP-2, IGF-1, kartogenin, or combinations thereof. In certain embodiments, for any the method disclosed herein compositions comprise a chondrogenic stimuli such as TGF-β- 1, 2, and 3, BMP-2, 4, 7, cartilage-derived morphogenetic protein-1 (CDMP-1), IGF-1, bFGF, SMAD-1, -2, -3, -4, -5, -6, -7, -8, EGF, PDGF, type II collagen, type IX collagen, cartilage-link protein, SOX5, SOX6, SOX9, MEF2C, Dlx5, Nkx2.5, Parathyroid hormone related peptide (PTHrP), Indian hedgehog signaling molecule (Ihh), Connective tissue growth factor (CTGF), or combinations thereof.

In certain embodiments, the Rho-kinase inhibitor and chondrogenic agent are contained in a matrix wherein the matrix optionally comprises collagen or a fibrin matrix optionally comprising factor XIII, factor XIIIα, fibrinogen, fibronectin, plasminogen, thrombin, aprotinin, TGF-β, or combinations thereof.

In certain embodiments, implanting or injecting a Rho-kinase inhibitor and a chondrogenic agent is in combination with thrombin and aprotinin and optionally TGF-β.

In certain embodiments, implanting or injecting factor XIII, factor XIIIα, fibrinogen, fibronectin, plasminogen, thrombin, aprotinin, TGF-β, or combinations thereof is locally at the area of desired cartilage growth, repair, or tissue replacement.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting fasudil, aprotinin, and a chondrogenic agent at the area of desired cartilage growth in an amount effective to generate the formation or repair of cartilage or tissue replacement.

In certain embodiments, the chondrogenic agent is transforming growth factor beta, dexamethasone, ascorbic acid or salt thereof, BMP-7, BMP-2, IGF-1, kartogenin, or combinations thereof. In certain embodiments, fasudil, aprotinin, and chondrogenic agent are contained in a matrix. In certain embodiments, the matrix optionally comprises collagen or a fibrin matrix optionally comprising factor XIII, factor XIIIα, fibrinogen, fibronectin, plasminogen, thrombin, aprotinin, TGF-β, or combinations thereof.

In certain embodiments, methods further comprise implanting or injecting factor XIII, factor XIIIα, fibrinogen, fibronectin, plasminogen, thrombin, aprotinin, TGF-β, or combinations thereof at the area of desired cartilage growth, repair, or tissue replacement.

In certain embodiments, this disclosure relates to methods of repairing or regenerating cartilage, minimizing the formation of fibrous tissue after microfracture, stimulating marrow, or preventing microfracture clot contraction after microfracture comprising puncturing a bone surface on an area of desired cartilage growth at a sufficient depth to expose bone marrow containing mesenchymal stromal cells which migrate into the bone surface or cartilage defect, and implanting or injecting a Rho-kinase inhibitor, aprotinin, and a chondrogenic agent at the area of desired cartilage growth in an amount effective to generate the formation or repair of cartilage or tissue replacement. In certain embodiments, the Rho-kinase inhibitor is fasudil, ripasudil, netarsudil, 4-(1-aminoethyl)-N-(pyridin-4-yl)cyclohexane-1-carboxamide (Y27632), or salt thereof.

In certain embodiments, the chondrogenic agent is transforming growth factor beta (TGF-β), dexamethasone, ascorbic acid or salt thereof, BMP-7, BMP-2, IGF-1, kartogenin, or combinations thereof. In certain embodiments, wherein Rho-kinase inhibitor, aprotinin, and chondrogenic agent are contained in a matrix. In certain embodiments, the matrix optionally comprises collagen or a fibrin matrix optionally comprising factor XIII, factor XIIIα, fibrinogen, fibronectin, plasminogen, thrombin, aprotinin, TGF-β, or combinations thereof. In certain embodiments, the method further comprises implanting or injecting factor XIII, factor XIIIα, fibrinogen, fibronectin, plasminogen, thrombin, aprotinin, TGF-β, or combinations thereof at the area of desired cartilage growth, repair, or tissue replacement.

In certain embodiments, this disclosure contemplates an injectable solution comprising any component or combination of components as provided herein.

In certain embodiments, this disclosure contemplates an implantable matrix or gel comprising any component or combination of components as provided herein. In certain embodiments, the matrix is biodegradable.

In certain embodiments, this disclosure contemplates uses of any a component or combination of components as provided herein in the production of a medicament for repairing or regenerating cartilage, minimizing the formation of fibrous tissue after microfracture, stimulating marrow, or preventing microfracture clot contraction after microfracture.

In certain embodiments, this disclosure relates to methods of cartilage and marrow stimulation. In certain embodiments methods disclosed herein can be applied to rotator cuff and meniscus repair. In certain embodiments, this disclosure relates to systems, methods, and formulations which can be applied to any musculoskeletal tissue repair augmented with marrow stimulation. In certain embodiments, this disclosure relates to improving cartilage repair. In certain embodiments, this disclosure relates to filling the bone defect surface, or tissue defect or cartilage defect.

In certain embodiments, methods disclosed herein include administering or injecting a liquid solution or implanting a matrix comprising a recombinant vector encoding SOX9, mesenchymal stromal cells (MSCs) comprising a recombinant vector encoding SOX9, or combinations thereof.

In some embodiments, the recombinant vector encoding SOX9 is capable of inducing mesenchymal stem cells (MSCs) to differentiate into chondrocytes. In certain embodiments, the vector encoding SOX9 further encodes a fusion protein of SOX9 and enhanced cell-penetrating peptide with a transduction domain to facilitate penetration of the cell membrane and get into cell nuclei. In certain embodiments, the transduction domain is selected from the group consisting of poly-arginine or poly-lysine sequence, e.g., 8 or greater continuous arginine or lysine, human immunodeficiency virus type 1 (HIV-1) Tat protein transduction domain (PTD), Antennapedia, herpes virus structural protein 22 (vp22), penetratin, etc. In certain embodiments, the transduction domain is fused to the N-termini or the C-termini of the SOX9 polypeptide.

In certain embodiments, compositions reported herein are administered to the microfracture site by loading components to a carrier matrix, such as a collagen membrane, and the matrix is placed and/or secured on the surface of microfracture sites during the procedure.

In certain embodiments, compositions reported herein are administered by directly injecting the composition into the synovial cavity of the microfracture in a patient.

In certain embodiments, this disclosure relates to methods for repairing cartilage damage such as to repair a fresh cartilage injury or in an aged subject/patient over the age of 50, 55, 60, or 65 years and to treat osteoarthritis derived from cartilage injury.

In certain embodiments, it is contemplated that the method halts or delays progression of cartilage injury and progression to osteoarthritis and delays the requirement of joint replacement in patients diagnosed with osteoarthritis.

In certain embodiments, the methods comprise creating a microfracture or performing other bone marrow stimulation techniques on a patient inflicted with cartilage damage; and administering a composition comprising components disclosed herein to the site of the microfracture. In certain embodiments, the method includes shaving or scraping the base of the defective cartilage location to induce bleeding at the desired location. In certain embodiments an arthroscopic awl or pick is used. In certain embodiments, small holes are made in the subchondral bone plate. In certain embodiment, an end of the awl is manually struck with a mallet to form the holes without penetrating or damaging the subchondral plate. In certain embodiments, the holes penetrate a vascularization zone and stimulate the formation of a fibrin clot containing MSCs and possibly other pluripotent stem cells.

In certain embodiments, methods of this disclosure include steps of drilling small holes deep into the subchondral bone marrow space, microfracture induces bleeding of bone marrow and forms clot at the surface of cartilage defect wherein MSCs contained in the bone marrow clot then differentiate into chondrocytes, osteocytes, muscle cycles, stromal cells, or fibroblasts. In certain embodiments, methods are used to reduce a percentage of MSCs to form stromal cells and fibroblasts and reducing the formation of fibrocartilage.

In certain embodiments, methods disclosed herein may be performed on a patient with musculoskeletal repairs, such as rotator cuff and meniscus tears or other injuries to joints, elbow, shoulder, femoral condyles, tibial plateau, patella, and ankle.

In certain embodiments, methods and compositions disclosed herein may be used in other medical procedures such as before, after, or during the performance of an osteotomy, microfracture, abrasion arthroplasty, autologous chondrocyte transplantation, mosaicplasty, autologous osteochondral graft, and arthroplasty. In certain embodiments, methods and compositions disclosed herein may be used when implanting an osteochondral allograft.

In certain embodiments, pharmaceutical preparations of the disclosure are preferably in a unit dosage form, and can be suitably packaged, for example in a box, blister, vial, bottle, sachet, ampoule or in any other suitable container (which can be properly labeled); optionally with one or more leaflets containing product information and/or instructions for use.

In certain embodiments, this disclosure contemplates kits comprising fasudil or other a Rho-kinase inhibitor as provided herein. In certain embodiments, the kit comprises: a) a first vial or first storage container containing a Rho-kinase inhibitor, and b) a second vial or second storage container having a second component as reported herein, e.g., thrombin, aprotinin, and/or TGF-β, and said kit further optionally contains a matrix as reported herein or a third vial or third storage container having a third component as reported herein and optionally comprising instructions for use thereof.

In certain embodiments, this disclosure contemplates a kit comprising a pharmaceutical composition comprising components disclosed herein and a container with a suitable diluent. Further components of the kit may be instructions for use, administration means, such as syringes, catheters, brushes, etc. (if the compositions are not already provided in the administration means) or other components necessary for use in medical (surgical) practice, such as substitute needles or catheters, extra vials or further wound cover means. In certain embodiments, the kit comprises a syringe housing the dry and stable hemostatic composition and a syringe containing the diluent (or provided to take up the diluent from another diluent container). The kits may contain a transfer device such a needle, syringe, cannula, capillary tube, pipette, or pipette tip. In certain embodiments, compositions may be contained in a storage container, dispensing container, sealed, or unsealed, such a vial, bottle, ampule, blister pack, or box. In certain embodiments, other agents may be contained in a storage container, sealed, or unsealed, such a vial, bottle, ampule, blister pack, or box.

### Marrow Stimulation Using Exogenous Agents

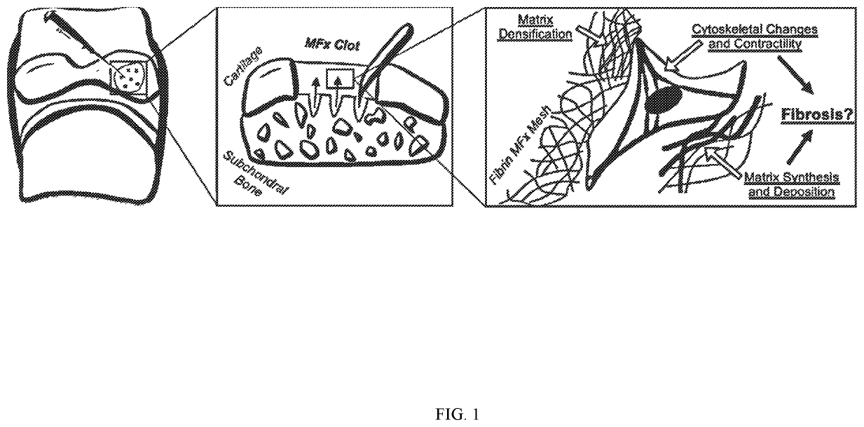

Articular cartilage is dense connective tissue that lines the surfaces of joints. Due to the complexities of joint motion, the tissue is often injured, either traumatically or by wear and tear. Unfortunately, many of these injuries lack the capacity to self-heal, and surgical intervention is commonly required. Microfracture (MFx) is a common repair technique and involves puncturing the subchondral bone, which recruits marrow elements that clot in the defect (FIG. 1). While short-term symptom relief is provided, the MFx clot often experiences inferior fibrous tissue formation, leaving the repair tissue and surrounding native cartilage susceptible to wear and subsequent deterioration to osteoarthritis. One way to improve these results is by implantation of chondrocytes (cartilage cells), or chondrocyte stem cell precursors, such as induced pluripotent chondrocytes, or augmentation of MFx with scaffolds and growth factors to improve chondrogenesis. Experiments were performed to understand the micro-scale environmental drivers of MFx fibrosis represents a strategy to improve the outcomes and augment MFx for the purpose of treating, preventing or delaying the onset of osteoarthritis.

Articular cartilage is a tissue with a dense composition of type II collagen and proteoglycans. The tissue is maintained by a relatively low concentration of chondrocytes, the morphologically round cells that preserve cartilage integrity. Cartilage injuries, whether traumatic or by wear-and-tear, alter tissue and chondrocyte homeostasis, and are unable to self-heal due to the avascularity of the tissue. To treat the mechanical irritation, discomfort, and pain caused by these injuries, surgeons often remove the damaged cartilage (chondroplasty). However, further damage to the injured site, and to the cartilage remaining after removal, often progresses in severity and leads to a vicious cycle of deterioration, motivating surgically induced repair.

MFx has been used to puncture the subchondral bone with an awl or drill, allowing marrow from the underlying trabecular structures to flow into the defect and clot (FIG. 1). Limited results are typically attributed to the formation of mechanically inferior fibrous tissue. Other contemplated restorative therapies include autologous chondrocyte implantation or matrix-induced autologous chondrocyte implantation (ACI, MACI). However, improved methods are needed.

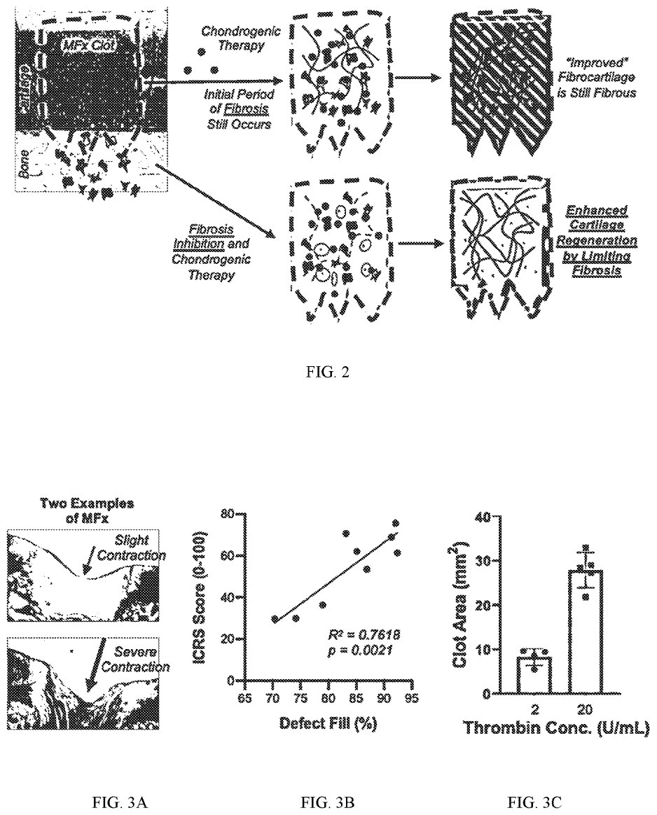

Marrow stimulation is contemplated for other musculoskeletal repairs, such as rotator cuff and meniscus. Thus, guiding marrow stimulation towards functional repair tissue is desirable. To improve outcomes, one can augment MFx with scaffolds, bioactive factors, and cells. Clinically, autologous matrix-induced chondrogenesis (AMIC) using a collagen membrane (types I/III) to cover the marrow clot in combination with exogenous agents is contemplated (FIG. 2). Contemplated agents include biologic (collagen, hyaluronic acid) and synthetic (polycaprolactone, polylactic acid) polymers, which release an array of chondrogenic factors, transforming growth factor beta 3 (TGF-β3) with the objective of improving properties so they are similar to hyaline cartilage.

### Clot Contraction

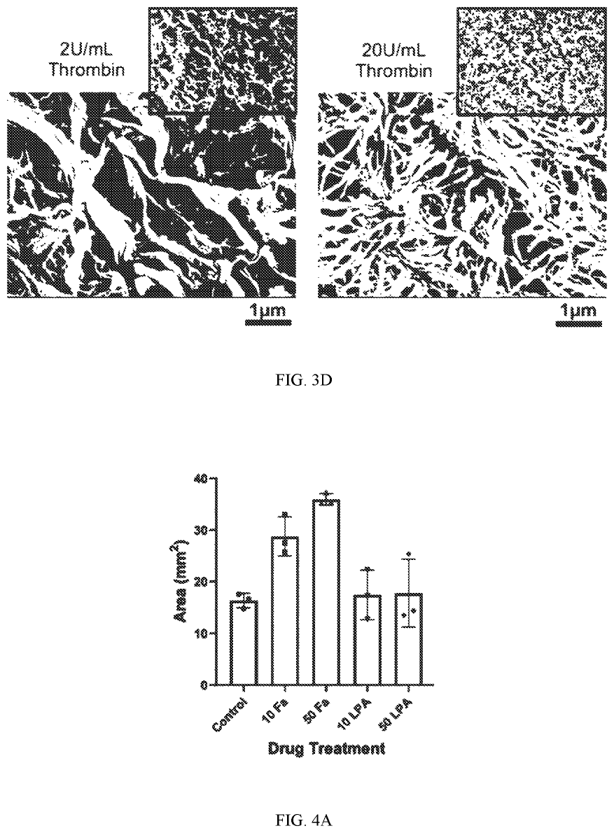

Experiments using MFx were performed in large animals, and it was observed that a reduction in the fill of the defect, i.e., a contraction resulted in a “blood clot”-like environment (FIG. 3A). Furthermore, the degree of contraction (reduced defect fill) led to heightened fibrosis and worse overall outcomes (FIG. 3B), indicating that a link exists between contraction and fibrosis. MFx clots were simulated in vitro (fibrin gels). The initial structural and mechanical properties were affected by the concentration of thrombin and influential on clots contract over time (FIG. 3C).

It is contemplated that one can fabricate fibrin gels with a combination of fibrinogen and thrombin. Marrow cells can be seeded within the fibrin network, and one can evaluate actin polymerization as densification typically occurs in the first 8 hours. After this initial period of contraction and densification, one can apply either a fibrin crosslinker (e.g. poly-stat) or fibrinolytic agents (e.g. plasmin) to stiffen or soften the network, respectively. Also contemplated are systems with a mixture of fibrinogen, e.g., 1-50 mg/mL and thrombin, e.g., 0.5-20 U/mL and other factors like calcium chloride and aprotinin.

### Live-Cell Cytoskeletal Reorganization and Fibrosis in Response to Dynamic Stimuli

It is contemplated that cells undergo initial cytoskeletal changes; thus, the role of cellular contractility (and specifically the Rho/ROCK pathway) on fibrosis is contemplated. The cellular tension generation via Rho/ROCK may be a driver of fibrosis in MFx clots; thus, inhibitors can prevent fibrosis.

Prior to seeding into the fibrin networks, marrow MSCs will undergo two transfections, one for vinculin (Vinculin-GFP plasmid transfection for focal adhesions) and one for actin (b-Actin-RFP lentivirus transfection). Thus, as vinculin and actin are expressed and organized in focal adhesions or stress fibers, respectively, one can actively monitor development in response to the initial densification and subsequent stiffening/softening. Colocalization of the focal adhesions to densified networks can be measured to elucidate how cells begin to place traction on the MFx clot. Furthermore, the degree of actin polymerization per cell can be quantified.

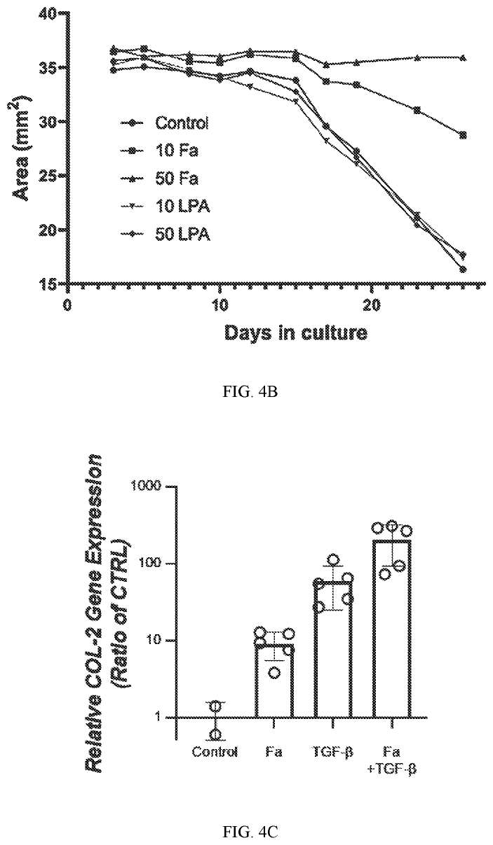

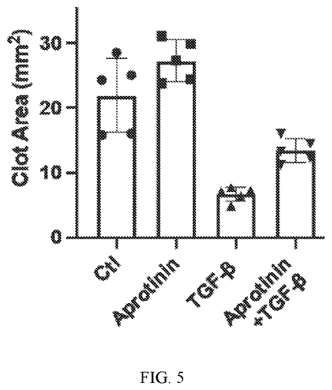

In addition to the chemical mediated changes to the environment, one can also alter the cellular machinery, to clarify the mechanisms that link cell contraction to fibrosis. Incorporating an inhibitor (Fasudil) and/or activator (lysophosphatidic acid [LPA]) of the Rho-ROCK pathway, allows one to evaluate the effects on the initial densification process, and the actin polymerization inhibitor cytochalasin D. Initial testing with Fasudil and LPA showed effects on whole clots (FIGS. 4A-4C) as well as changes in actin morphology and densification.

1. A method of repairing damaged cartilage comprising administering an amount effective of fasudil at the area of desired cartilage growth to a subject in need thereof.

2. The method of claim 1, wherein administering fasudil is in combination with thrombin and/or aprotinin.

3. The method of claim 1, wherein administering fasudil is in combination with transforming growth factor-beta.

4. The method of claim 1, wherein administering is injecting a liquid solution or implanting matrix comprising fasudil and transforming growth factor-beta.

5. The method of claim 4, wherein the liquid solution further comprises thrombin, aprotinin, factor XIII, factor XIIIα, fibrinogen, fibronectin, plasminogen, or combinations thereof.

6. The method of claim 4, wherein the matrix is a collagen matrix or a fibrin matrix.

7. The method of claim 4, wherein the matrix further comprises thrombin, aprotinin, factor XIII, factor XIIIα, fibrinogen, fibronectin, plasminogen, or combinations thereof.

8. A method of repairing damaged cartilage comprising
puncturing a bone surface on an area of desired cartilage growth at a sufficient depth to expose bone marrow containing mesenchymal stromal cells which migrate into the bone surface or cartilage defect, and
administering an amount effective of fasudil at the area of desired cartilage growth.

9. A method of minimizing the formation of fibrous tissue after microfracture comprising
puncturing a bone surface on an area of desired cartilage growth at a sufficient depth to expose bone marrow containing mesenchymal stromal cells which migrate into the bone surface or cartilage defect, and
administering an amount effective of fasudil at the area of desired cartilage growth.

10. The method of claim 9, wherein administering fasudil is in combination with thrombin and/or aprotinin.

11. The method of claim 9, wherein administering fasudil is in combination with transforming growth factor-beta.

12. The method of claim 9, wherein administering is injecting a liquid solution or implanting matrix comprising fasudil and transforming growth factor-beta.

13. The method of claim 12, wherein the liquid solution further comprises thrombin, aprotinin, factor XIII, factor XIIIα, fibrinogen, fibronectin, plasminogen, or combinations thereof.

14. The method of claim 12, wherein the matrix is a collagen matrix or a fibrin matrix.

15. The method of claim 12, wherein the matrix further comprises thrombin, aprotinin, factor XIII, factor XIIIα, fibrinogen, fibronectin, plasminogen, or combinations thereof.

16. A composition comprising of fasudil and transforming growth factor-beta.

17. The composition of claim 16 further comprising thrombin and/or aprotinin.

18. The composition of claim 16 in the form of an injectable solution.

19. The composition of claim 16, in the form of a gel or polymer matrix.

20. The composition of claim 19, wherein the polymer matrix is a collagen matrix or a fibrin matrix.
